(19) United States
(12) Patent Application Publication
(10) Pub. No.: US 2024/0269155 A1
(43) Pub. Date: Aug. 15, 2024

(54) METHODS AND USES OF BORON COMPOUNDS IN THE TREATMENT OF NONTUBERCULOUS MYCOBACTERIUM INFECTIONS AND PHARMACEUTICAL COMPOSITIONS FOR TREATMENT OF SAME

(71) Applicant: SHANGHAI MICURX PHARMACEUTICALS CO., LTD., Shanghai (CN)

(72) Inventors: Xinghai Wang, Shanghai (CN); Shicong Liu, Shanghai (CN); Jinqian Liu, Fremont, CA (US); Mikhail Fedorovich Gordeev, Castro Valley, CA (US); Zhengyu Yuan, Palo Alto, CA (US)

(21) Appl. No.: 18/605,432

(22) Filed: Mar. 14, 2024

Related U.S. Application Data
(63) Continuation of application No. PCT/CN2023/101603, filed on Jun. 21, 2023.
(60) Provisional application No. 63/366,910, filed on Jun. 23, 2022.

(51) Int. Cl. A61K 31/69; A61P 31/04

(52) U.S. Cl. CPC A61K 31/69; A61P 31/04

(57) ABSTRACT

Provided herein are organoboron compounds of Formula I, or salts thereof, and pharmaceutical compositions and the use of organoboron compounds and pharmaceutical compositions for the treatment of nontuberculous Mycobacterium infections.

### FIELD

Provided herein are organoboron compounds for the treatment of nontuberculous Mycobacterium infections and pharmaceutical compositions for treatment of the same.

### BACKGROUND

Nontuberculous Mycobacteria (NTM) pulmonary disease (NTM-PD) is a severe progressive illness caused by certain Mycobacterial species that can require complicated treatment with multiple anti-mycobacterial drugs or combinations of such antibiotics for more than 12 months (as reviewed, for example, by Daley et al. in Clin. Infect. Dis. 2020 Aug. 15; 71(4): 905-913). NTM refers to all Mycobacterium species except Mycobacterium tuberculosis complex and Mycobacterium leprae. There are over 190 species of NTM bacteria found to-date, and while most are parasitic bacteria, only a few are conditional pathogens that can cause an infection in a human. However, in recent years, with the increase of patients with acquired immunodeficiency syndrome and immunosuppressive population, there has been a worldwide increase in the incidence rate and prevalence of NTM disease. Moreover, the resistance of NTM to antibiotics is increasing. The resistance rate is very high in relapsed patients in particular, which can cause difficulties for clinical treatment. Mortality of NTM-PD is higher than that attributable to Mycobacterium tuberculosis (MTB) due to inappropriate treatment and high rates of therapy failure.

NTM flora is divided into four groups by Runyon classification according to growth temperature, growth rate, colony morphology and the relationship between pigment production and light reaction. The first three groups are slow-growing mycobacteria, while the fourth group is a fast-growing mycobacteria. Group I are photochromogens and is mainly composed of M. kansasii, M. marinum and M. simide, while Group II are scotochromogens and mainly composed of M. scrofulaceum, M. gordonae and M. szulgai. Group III is non-photochromogens and include M. avium complex (MAC), M. haemophilus. ulcerans, M. xenopi. M. malmoense, M. terrae and M. gasteri. Group IV, the rapidly growing mycobacteria (RGM), include M. abscessus complex (MABC). M. fortuitum, M. chelonae, M. margeritense, M. peregrinum, M. smegmatis and M. vaccae.

Since NTM comprise a group of bacteria that causes serious lung infections, treatment is usually complex and requires an extended period of treatment. Furthermore, because most NTM is inherently resistant to standard anti-tuberculosis drugs, and different species exhibit varying resistance phenotypes, the available drugs and programs for treatment are limited.

For many patients with bacterial infections who require long-term treatment with an antibiotic, an oral formulation is the most appropriate choice. Advantages of oral delivery over an intravenous route include the absence of cannula-related infections, a lower drug cost, and a reduction in hospital costs (such as the need for a health professional and equipment to administer intravenous antibiotics). Oral therapy is also particularly important to ensure the patient compliance for those who require long-term treatment. For example, the treatment of Mycobacterium avium and Mycobacterium abscessus infection usually takes several months.

Thus, there exists a need for new therapeutic agents with new modes of action, potent activity against drug-resistant isolates, little side effects, and convenient oral administration.

### SUMMARY

Provided herein are boron compounds and pharmaceutical compositions thereof for the treatment of nontuberculous mycobacterial infections.

The boron-organic compound shown below belongs to a class of antibiotics with high antibacterial activity, including Gram-negative and Gram-positive microorganisms, as well as against mycobacteria.

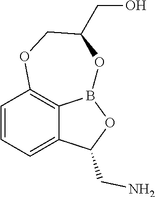 [SMILES: NC[C@H]1OB2O[C@H](CO)COc3cccc1c32]

As described in U.S. Pat. No. 8,530,452, this tricyclic boron compound is particularly active against Gram-negative bacteria such as Pseudomonas aeruginosa, Acinetobacter baumannii, Escherichia coli, and Klebsiela pneumoniae. However, activity of this compound against mycobacteria was not reported. In fact, mycobacterial cell envelope is very different from typical Gram-positive and Gram-negative bacteria, and it cannot be assumed that an antibiotic will possess antibacterial potency in both Gram-negative bacteria and mycobacteria. As described herein, it has been discovered that a salt form of this boron compound unexpectedly exhibits activity against mycobacteria. Moreover, as described below, this compound exhibits only moderate oral bioavailability. In contrast, certain prodrugs described herein exhibit enhanced oral bioavailability and improved systemic exposure critical for the pathogen eradication. Certain compounds described herein exhibit important advantageous dual therapeutic properties: potency against mycobacteria and oral bioavailability. This represent a significant advancement over the majority of antibiotics, such as cephalosporins, that can only be given via intravenous administration in a hospital setting.

In one aspect, provided herein is a method of treating a nontuberculous mycobacterial infection comprising the administration of a therapeutically effective amount of a compound of Formula (I):

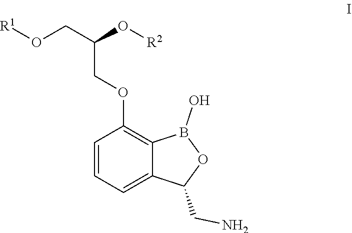

or pharmaceutically acceptable salts thereof to a mammal in the need of therapy;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; and
R² is selected from C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; or
R¹ and R² taken together form a heterocyclic group selected from 1,3-dioxane, 2-C₁₋₆-alkyl-1,3-dioxane, 2,2-di(C₁₋₆-alkyl)-1,3-dioxane, 2-methyl-1,3-dioxane, 2-aryl-1,3-dioxane, 2-(2-carboxyphenyl)-1,3-dioxane, 2-(4-carboxyphenyl)-1,3-dioxane, and 2-C₁₋₆-alkylOC(═O)-1,3-dioxane; each of which is optionally substituted with one to four R³;
R³ at each occurrence is independently selected from the group consisting of halo, hydroxy, C₁-C₆ alkyl, C₃-C₆ cycloalkyl, C₁-C₆ alkoxy, aryl, and heteroaryl; or
two R³ groups when attached to adjacent carbons are taken together with the carbons to which they are attached to form a fused C₃-C₆ cycloalkyl; or
two R³ groups when attached to the same carbon are taken together with the carbon to which they are attached to form a spiro C₃-C₆ cycloalkyl;
wherein each R³ is optionally independently substituted with one to three halo, hydroxy, or C₁-C₃ alkyl; or
the hydrogen in the —OH attached to the boron atom is not present and R² in the compound of Formula I, together with the oxygen attached to the boron atom, form a compound of Formula II. Therefore, in another aspect, provided herein is a method of treating a nontuberculous mycobacterial infection comprising the administration of a therapeutically effective amount of a compound of Formula II:

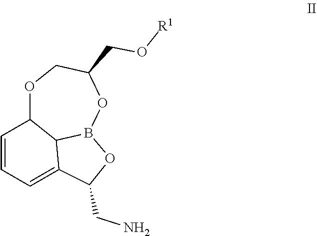

or a pharmaceutically acceptable salt thereof to a mammal in the need of therapy;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl.

In another aspect, provided herein is a use of the compounds of Formula I or Formula II or a pharmaceutically acceptable salt thereof in the manufacturing of a medicament for treating nontuberculous mycobacterial infections.

In yet another aspect, provided herein is a pharmaceutical composition comprising a compound of Formula I or Formula II, or a pharmaceutically acceptable salt thereof, and at least one pharmaceutically acceptable carrier, for the treatment of nontuberculous mycobacterial infections.

Nontuberculous mycobacteria include, but not limited to, Mycobacterium scrofulaceum, Mycobacterium gordonae, Mycobacterium avium, Mycobacterium abscessus, Mycobacterium intercelleulare, Mycobacterium fortuitum, Mycobacterium peregrinum, Mycobacterium smegmatis, and Mycobacterium massiliense.

### BRIEF DESCRIPTION OF FIGURES

FIG. 1 is a graph for the count of the colony-forming units (CFU) in the lungs of mice 14 days following administration of the compound of Example 4 (10 mg/kg subcutaneously, daily), GSK656 (10 mg/kg subcutaneously, daily), linezolid (100 mg/kg daily by oral gavage), or clarithromycin (200 mg/kg daily by oral gavage) as described in Example 7.

FIG. 2A is an image of an H&E stained tissue section of a mice uninfected with M. abscessus CIP108297 in the M. abscessus mouse lung infection model described below.

FIG. 2B is an image of an H&E stained tissue section of a mice infected with M. abscessus CIP108297, but untreated, after 14 days, in the M. abscessus mouse lung infection model described below.

FIG. 2C is an image of an H&E stained tissue section of a mice infected with M. abscessus CIP108297, and treated with the compound of Example 4, after 14 days, in the M. abscessus mouse lung infection model described below.

### DETAILED DESCRIPTION

Unless otherwise stated, the following terms used in the specification and claims have the meanings given below:

The terms alkyl, alkenyl, etc. refer to both straight and branched groups, but reference to an individual radical such as “propyl” embraces only the straight chain radical and a branched chain isomer such as “isopropyl” embraces only the branched chain isomer. The alkyl, alkenyl, etc. group may be optionally substituted with one, two, or three substituents selected from the group consisting of halo, aryl, Het¹, or Het². Representative examples include, but are not limited to, difluoromethyl, 2-fluoroethyl, trifluoroethyl. —CH═CH-aryl, —CH═CH-Het¹, —CH₂-phenyl, and the like.

The term “cycloalkyl” refers to a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, aryl, Het¹, or Het².

The term “heteroalkyl” refers to an alkyl or cycloalkyl group, as defined above, having a substituent containing a heteroatom selected from N, O, or S(O)ₙ, where n is an integer from 0 to 2, including, hydroxy (OH), C₁₋₄alkoxy, amino, thio (—SH), and the like. Representative substituents include —NRₐR_{b}, —ORₐ, or —S(O))ₙ—R_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is C₁₋₄alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b} where Rₐ and R_{b} are as defined above. Representative examples include, but are not limited to 2-methoxyethyl (—CH—CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term aryl refers to phenyl, biphenyl, or naphthyl, optionally substituted with 1 to 3 substituents independently selected from halo, —C₁₋₄alkyl, —OH, —OC₁₋₄alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or —δC₁₋₄alkyl.

Het¹ at each occurrence is independently a C-linked 5- or 6-membered heterocyclic ring having 1 to 4 heteroatoms selected from the group consisting of oxygen, nitrogen, and sulfur within the ring. Het2 at each occurrence is independently a N-linked 5 or 6 membered heterocyclic ring having 1 to 4 nitrogen and optionally having one oxygen or sulfur within the ring. “Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentancpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “tautomers” means two or more forms or isomers of an organic compound that could be interconverted into each other via a common chemical reaction called tautomerization, generally analogous to that described by Smith et al., in Advanced Organic Chemistry. 2001, 5th Ed. NY: Wiley Interscience., pp. 1218-1223. The concept of tautomerizations is called tautomerism. The tautomerism may be accompanied by a change from a ring structure to an open structure, as observed, for example, for interconversion between the cyclic pyran form and the open chain form of glucose via formation and breaking of a C—O bond. The degree of tautomerism is often affected by a solvent effect, such as a hydration with water, and the media acidity. A related process concerning cyclic boron compounds may involve formation and breaking of a B—O bond as exemplified below:

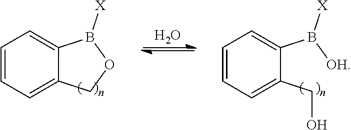

“Treating” or “treatment” of a disease includes:
(1) preventing the disease, i.e., causing the clinical symptoms of the disease not to develop in a mammal that may be exposed to or predisposed to the disease, but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Prodrug” means any compound which releases an active parent drug in vivo when such prodrug is administered to a mammalian subject. Prodrugs of the compounds described herein are prepared by modifying functional groups present in a compound described herein in such a way that the modifications may be cleaved in vivo to release the parent compound. Prodrugs include compounds described herein wherein a hydroxy, sulfhydryl, amido or amino group in the compound is bonded to any group that may be cleaved in vivo to regenerate the free hydroxyl, amido, amino, or sulfhydryl group, respectively.

“Patient” and “patients” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the patient is a human.

### Illustrative Embodiments

In aspect, provided herein is a method for treating nontuberculous mycobacterial infections comprising administrating a therapeutically effective amount of a compound of Formula (I):

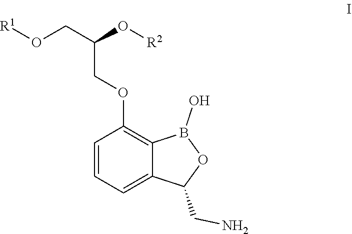

or a pharmaceutically acceptable salt thereof to a patient in need of treatment;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; and
R² is selected from C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; or
R¹ and R² taken together form a heterocyclic group selected from 1,3-dioxane, 2-C₁₋₆-alkyl-1,3-dioxane, 2,2-di(C₁₋₆-alkyl)-1,3-dioxane, 2-methyl-1,3-dioxane, 2-aryl-1,3-dioxane, 2-(2-carboxyphenyl)-1,3-dioxane, 2-(4-carboxyphenyl)-1,3-dioxane, and 2-C₁₋₆-alkylOC(═O)-1,3-dioxane; each of which is optionally substituted with one to four R³;
R³ at each occurrence is independently selected from the group consisting of halo, hydroxy, C₁-C₆ alkyl, C₃-C₆ cycloalkyl, C₁-C₆ alkoxy, aryl, and heteroaryl; or
two R³ groups when attached to adjacent carbons are taken together with the carbons to which they are attached to form a fused C₃-C₆ cycloalkyl; or
two R³ groups when attached to the same carbon are taken together with the carbon to which they are attached to form a spiro C₃-C₆ cycloalkyl;
   wherein each R³ is optionally independently substituted with one to three halo, hydroxy, or C₁-C₃ alkyl; or
the hydrogen in the —OH attached to the boron atom is not present and R² in the compound of Formula I, together with the oxygen attached to the boron atom, form a compound of Formula II. Therefore in another aspect, provided herein is a method of treating a nontuberculous mycobacterial infection comprising the administration of a therapeutically effective amount of a compound of Formula II:

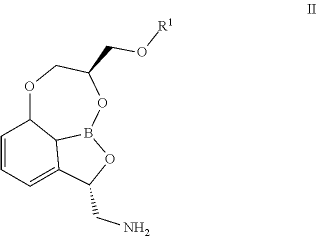

or a pharmaceutically acceptable salt thereof to a patient in need of treatment;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl.

In some embodiments, the method for treating nontuberculous mycobacterial infections comprising administrating a therapeutically effective amount of a compound of Formula (I) or a pharmaceutically acceptable salt thereof to a patient in need of treatment, wherein R¹ is C₁₋₄alkyl-C(═O)— and R² is C₁₋₄alkyl-C(═O)—.

In another aspect, provided herein is a use of a compound of Formula I or Formula II or a pharmaceutically acceptable salt thereof in the manufacturing of a medicament for treating nontuberculous mycobacterial infections.

Nontuberculous mycobacteria include, but are not limited to, Mycobacterium scrofulaceum, Mycobacterium gordonae, Mycobacterium avium, Mycobacterium abscessus, Mycobacterium intercelleulare, Mycobacterium fortuitum, Mycobacterium peregrinum, Mycobacterium smegmatis, and Mycobacterium massiliense.

Compounds of Formula I or Formula II can be administrated in the free base form thereof, or also in the form of salts and/or hydrates. In some embodiments, compounds of Formula I or Formula II are administrated in the form of a hydrochloride salt thereof.

Compound of Formula I or Formula II or pharmaceutically acceptable salts thereof can be administrated via various administration routes, including but not limited to, a route selected from orally, parenterally, intraperitoneally, intravenously, intraarterially, transdermally, sublingually, intramuscularly, rectally, transbuccally, intranasally, via inhalation, vaginally, intraoccularly, via local administration, subcutaneously, intraadiposally, intraarticularly, intraperitoneally and intrathecally. In a certain embodiment, the administration is oral.

The amount of a compound of Formula I or Formula II or a pharmaceutically acceptable salts thereof can be determined according to the severity of the disease, the response of the disease, any treatment-related toxicity, and/or the age and health status of the patient. In some embodiments, the amount of a compound of Formula I or Formula II or a pharmaceutically acceptable salt thereof is 10-1000 mg. In some embodiments, the amount of a compound of Formula I or Formula II or a pharmaceutically acceptable salt thereof is 100-600 mg. In some embodiments, the amount of a compound of Formula I or Formula II or a pharmaceutically acceptable salt thereof is 200-400 mg.

A compound of Formula I or Formula II or a pharmaceutically acceptable salt thereof can be administrated one or more times daily. In some embodiments, a compound of Formula I or Formula II or a pharmaceutically acceptable salt thereof is administrated once per day in the form of a single dosage. In one embodiment, the compound is administered twice per day in the form of a single dosage. In one embodiment, the compound is administered twice per day in a single dosage suitable for an oral solid formulation.

In another aspect, provided herein is a use of a compound of Formula I or Formula II or a pharmaceutically acceptable salt thereof in the manufacturing of a medicament for treating nontuberculous mycobacterial infections. Nontuberculous mycobacteria include, but are not limited to, Mycobacterium scrofulaceum, Mycobacterium gordonae, Mycobacterium avium, Mycobacterium abscessus, Mycobacterium intercelleulare, Mycobacterium fortuitum, Mycobacterium peregrinum, Mycobacterium smegmatis, and Mycobacterium massiliense.

In some embodiments, the pharmaceutical compositions are formulations suitable for oral administration, which include, but are not limited to tablets, capsules, dusts, granulates, drip pills, pastes, powders and the like. In a preferred embodiment tablets and capsules are used. The tablets can be common tablets, dispersible tablets, effervescent tablets, sustained release tablets, controlled release tablets or enteric coated tablets, and the capsules can be common capsules, sustained release capsules, controlled release capsules or enteric coated capsules. The oral formulation can be prepared with well-known pharmaceutically acceptable carriers in the art by conventional methods. The pharmaceutically acceptable carriers include bulking agents, absorbing agents, wetting agents, binding agents, disintegrating agents, lubricants and the like. The bulking agents include starch, lactose, mannitol, microcrystalline cellulose or the like; the absorbing agents include calcium sulfate, calcium hydrogen phosphate, calcium carbonate or the like; the wetting agents include water, ethanol or the like; the binding agents include hydroxypropyl methylcellulose, povidone, microcrystalline cellulose or the like; the disintegrating agents include cross-linked carboxymethyl cellulose sodium, crospovidone, surfactants, low-substituted hydroxypropyl cellulose or the like; the lubricants include magnesium stearate, talc powder, polyethylene glycol, sodium dodecylsulfate, talc powder or the like. The pharmaceutical excipients also include colorants, sweetening agents and the like.

In one embodiment, the pharmaceutical composition is a solid formulation suitable for oral administration. For example, the composition can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In a certain embodiment, the pharmaceutical composition is a capsule.

In preparing a formulation, it may be necessary to mill the active compound to provide the appropriate particle size prior to combine with the other ingredients. If the active compound is substantially insoluble, it is ordinarily milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound as described herein. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above.

In one embodiment, a pharmaceutical composition for treating a nontuberculous mycobacterial infection is formulated into a single dosage form. In one embodiment, the single dosage form contains 10 mg-1000 mg of a compound of Formula I or Formula II or a pharmaceutically acceptable salt thereof. In some embodiments, the single dosage form contains 100 mg-600 mg of a compound of Formula I or Formula II or a pharmaceutically acceptable salt thereof. In one embodiment, the single dosage form contains 200 mg-400 mg of a compound of Formula I or Formula II or a pharmaceutically acceptable salt, preferably 200 mg-400 mg of a compound of Formula I or Formula II or a pharmaceutically acceptable salt.

The tablets or pills as described herein may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, the administration is continuously administered for 4-18 months, and preferably for 4-6 months.

In another embodiment, the pharmaceutical composition is administrated in combination with other antibiotics that are currently used for management of NTM infections, such as, amikacin, clarithromycin, azithromycin, or ciprofloxacin.

Herein, unless indicated otherwise, the dosages and ranges provided therein are based on the molecular weight of the free base form of the compound of Formula I or Formula II.

A preferred compound of Formula II is:

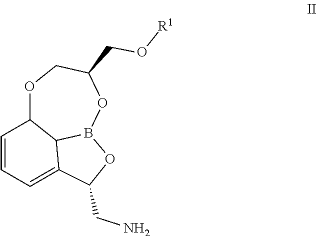

or a pharmaceutically acceptable salt thereof.

Additional preferred compounds of Formula I include structures below,

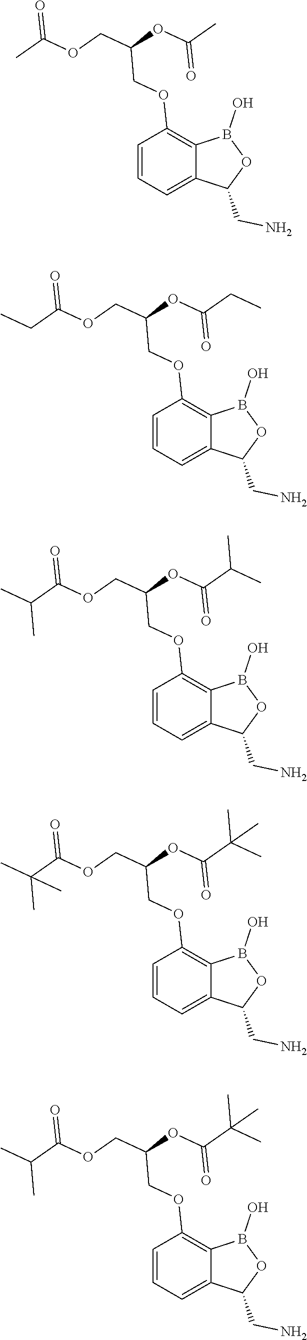 [SMILES: CC(=O)OC[C@H](COc1cccc2c1B(O)O[C@@H]2CN)OC(C)=O ; CCC(=O)OC[C@H](COc1cccc2c1B(O)O[C@@H]2CN)OC(=O)CC ; CC(C)C(=O)OC[C@H](COc1cccc2c1B(O)O[C@@H]2CN)OC(=O)C(C)C ; CC(C)(C)C(=O)OC[C@H](COc1cccc2c1B(O)O[C@@H]2CN)OC(=O)C(C)(C)C ; CC(C)C(=O)OC[C@H](COc1cccc2c1B(O)O[C@@H]2CN)OC(=O)C(C)(C)C]

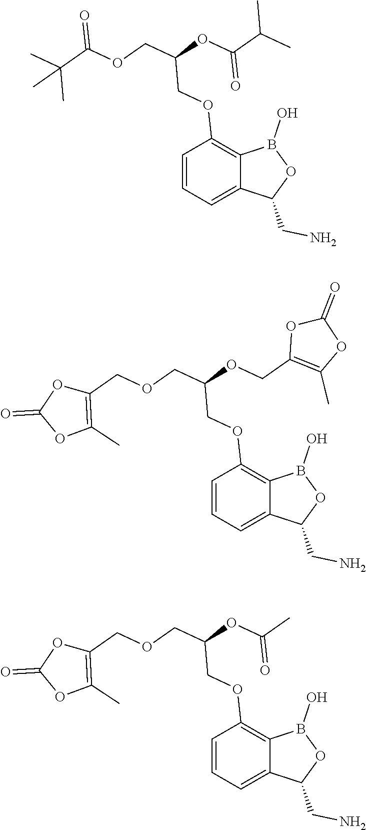 [SMILES: CC(C)C(=O)O[C@H](COC(=O)C(C)(C)C)COc1cccc2c1B(O)O[C@@H]2CN ; Cc1oc(=O)oc1COC[C@H](COc1cccc2c1B(O)O[C@@H]2CN)OCc1oc(=O)oc1C ; CC(=O)O[C@H](COCc1oc(=O)oc1C)COc1cccc2c1B(O)O[C@@H]2CN]

or a pharmaceutically acceptable salt thereof.

Preferred compounds of Formula II include structures below,

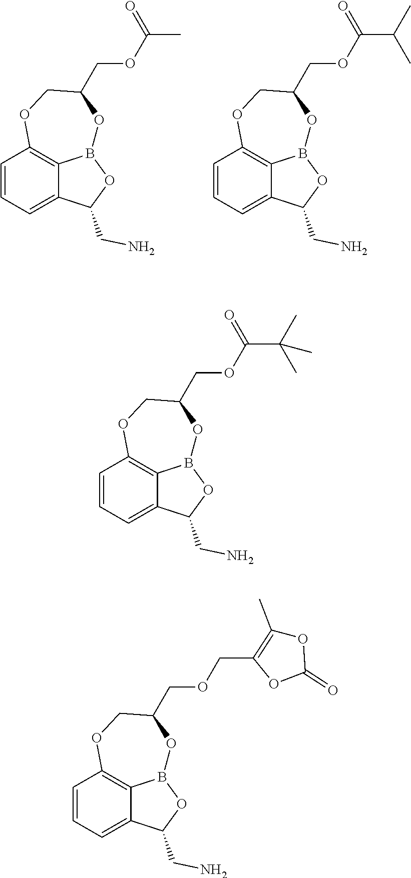 [SMILES: CC(=O)OC[C@@H]1COc2cccc3c2B(O1)O[C@@H]3CN ; CC(C)C(=O)OC[C@@H]1COc2cccc3c2B(O1)O[C@@H]3CN ; CC(C)(C)C(=O)OC[C@@H]1COc2cccc3c2B(O1)O[C@@H]3CN ; Cc1oc(=O)oc1COC[C@@H]1COc2cccc3c2B(O1)O[C@@H]3CN]

or a pharmaceutically acceptable salt thereof.

In some embodiments, the pharmaceutically acceptable salt form of compounds of Formulas I or II is a hydrochloride salt.

### EXAMPLES

Embodiments described in the following examples, which are meant to illustrate and not limit the scope of this disclosure. Common abbreviations well known to those with ordinary skills in the synthetic art are used throughout. ¹H NMR spectra were recorded on 300 MHz instrument in DMSO-d₆ unless specified otherwise. Mass-spectroscopy data for a positive ionization method are provided. Chromatography is silica gel chromatography unless specified otherwise. TLC is thin-layer chromatography. HPLC is reverse-phase HPLC. Unless specified otherwise, all reagents were either from commercial sources, or made by conventional methods described in available literature.

### Example 1. (2S)-3-Acetoxy-1-{[(3S)-3-(aminomethyl)-1-hydroxy-1,3-dihydrobenzo[2,1-c][1,2]oxaborol-7-yl]oxy}propan-2-yl acetate hydrochloride

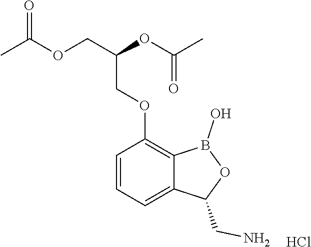 [SMILES: CC(=O)OC[C@H](COc1cccc2c1B(O)O[C@@H]2CN)OC(C)=O ; Cl]

### Scheme for Preparation of the Compound of Example 1

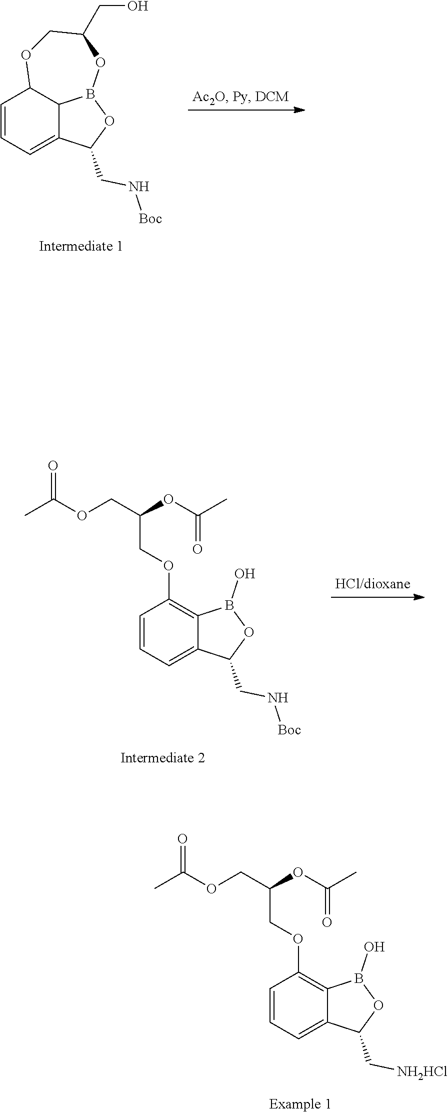

Intermediate 2. To a solution of Intermediate 1 (60 mg, 0.26 mmol, prepared as described in US Application US 2013/0165411) and pyridine (31 uL, 0.33 mmol) in DCM (2 mL) was added dropwise Ac₂O (32 uL, 0.33 mmol), and the mixture was stirred for 2 hours. After completion of the reaction, the solvent was removed by concentration, and the residue was purified on prep-HPLC to give Intermediate 2 (25 mg). MS (m/z): 438 [M+H].

Example 1. Intermediate 2 was dissolved in dioxane solution of 5M HCl (2 mL) at room temperature, and the mixture was stirred for 1 hour. Thereafter, the mixture was lyophilized to afford the compound of Example 1 (16.9 mg) as a light-yellow powder. MS (m/z): 338 [M+H]. ¹H NMR: (400 MHZ, D₂O): 7.48 (t, J=8.0 Hz, 1H); 7.01 (d, J=7.6 Hz, 1H); 6.92 (dd, J=12.0, 8.0 Hz, 1H); 5.35 (dd, J=7.4, 3.0 Hz, 1H); 4.35˜4.17 (m, 5H); 3.73˜3.60 (m, 1H); 3.56˜3.51 (m, 2H); 3.08˜3.01(m, 1H); 1.99 (s, 3H), 1.98 (s, 3H).

The compounds below were synthesized following the procedures described for Example 1.

| Ex- | | | [M + | |
|---|---|---|---|---|
| ample | Structure | Chemical Name | H]+ | ¹H NMR |
| 2 | 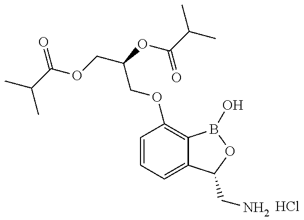 [SMILES: CC(C)C(=O)OC[C@H](COc1cccc2c1B(O)O[C@@H]2CN)OC(=O)C(C)C ; Cl] | (2S)-1-{[(3S)-3-(Aminomethyl)- 1-hydroxy-1,3-dihydrobenzo[2,1- c][1,2]oxaborol-7-yl]oxy}-3-[(2- methylpropanoyl)oxy]propan-2-yl 2-methylpropanoate hydrochloride | 394 | ¹H NMR (400 MHz, MeOD, ppm): δ 7.17 (t, J = 7.7 Hz, 1H), 6.79 (d, J = 7.4 Hz, 1H), 6.71 (d, J = 8.0 Hz, 1H), 5.38 (dd, J = 6.3, 3.9 Hz, 1H), 5.04 (s, 1H), 4.53 (dd, J = 11.9, 3.4 Hz, 1H), 4.38 (dd, J = 11.9, 6.8 Hz, 1H), 4.19 (t, J = 4.0 Hz, 2H), 3.31 (s, 2H), 3.24-3.18 (m, 1H), 3.04 (dd, J = 12.5, 3.9 Hz, 1H), 2.55 (dt, J = 13.2, 6.4 Hz, 2H), 1.15 (t, J = 6.8 Hz, 12H) |
| 3 | 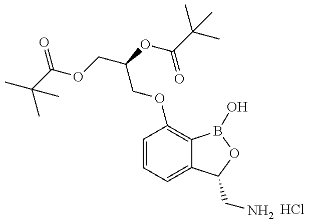 [SMILES: CC(C)(C)C(=O)OC[C@H](COc1cccc2c1B(O)O[C@@H]2CN)OC(=O)C(C)(C)C ; Cl] | (2S)-3-[(2,2- Dimethylpropanoyl)oxy]-1- {[(3S)-3-(aminoethyl)-1- hydroxy-1,3-dihdyrobenzo[2,1- c][1,2]oxaborol-7-yl]oxy}propan- 2-yl 2,2-dimethylpropanoate hydrochloride | 422 | ¹H NMR (300 MHz, DMSO-d₆, ppm): δ 8.63 (s, 1H), 7.40 (t, J = 7.8 Hz, 1H), 7.01 (d, J = 7.5 Hz, 1H), 6.85 (d, J = 8.1 Hz, 1H), 5.30 (s, 1H), 4.98 (dd, J = 6.6, 3.6 Hz, 1H), 4.43 (dd, J = 12.0, 3.1 Hz, 1H), 4.35-4.18 (m, 3H), 2.96 (dd, J = 13.2, 3.9 Hz, 1H), 2.62 (dd, J = 13.3, 6.8 Hz, 1H), 1.12 (s, 9H), 1.09 (s, 9H) |
| 6 | 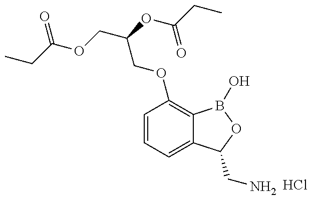 [SMILES: CCC(=O)OC[C@H](COc1cccc2c1B(O)O[C@@H]2CN)OC(=O)CC ; Cl] | (2S)-1-{[(3S)-3-(aminomethyl)-1- hydroxy-1,3-dihydrobenzo[2,1- c][1,2]oxaborol-7-yl]oxy}-3-[(2- methylpropanoyl)oxy]propan-2-yl propanoate hydrochloride | 366 | ¹H NMR (400 MHz, D₂O, ppm): 7.24 (d, J = 7.2 Hz, 1H); 6.86-6.78 (m, 2H); 5.40 (s, 1H); 5.10 (s, 1H); 4.43-4.19 (m, 4H); 3.33-3.30 (m, 1H); 3.12-3.08 (m, 1H); 2.35-2.68 (m, 4H); 1.01-0.97 (m, 6H) |

### Example 4. [(2S,6R)-2-(Aminomethyl)-4-bora-3,5,8-trioxatricyclo[7.3.1.04,13]trideca-1(12),10-dien-6-yl]methanol hydrochloride

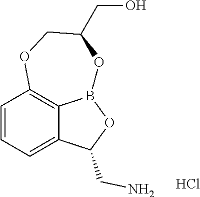 [SMILES: NC[C@H]1OB2O[C@H](CO)COc3cccc1c32 ; Cl]

The compound of Example 4 was prepared according to the methods described in US Application US 2013/0165411.

### Example 5. [(2S,6S)-2-(Aminomethyl)-4-bora-3,5,8-trioxatricyclo[7.3.1.04,13]trideca-1(12),9(13),10-trien-6-yl]methyl 2-methylpropanoate hydrogen chloride

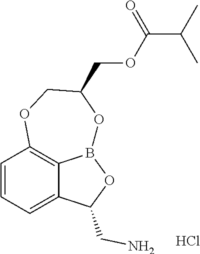 [SMILES: CC(C)C(=O)OC[C@@H]1COc2cccc3c2B(O1)O[C@@H]3CN ; Cl]

The compound of Example 5 was prepared according to the methods described in US Application US 2013/0165411.

### Example 7. [(2S,6S)-2-(aminomethyl)-4-bora-3,5,8-trioxatricyclo[7.3.1.04,13]trideca-1(12),9(13),10-trien-6-yl]methyl acetate hydrogen chloride

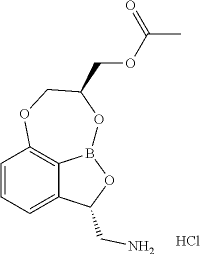 [SMILES: CC(=O)OC[C@@H]1COc2cccc3c2B(O1)O[C@@H]3CN ; Cl]

The compound of Example 7 was prepared according to the methods described in US Application US 2013/0165411.

### Example 8. [(2S,6S)-2-(aminomethyl)-4-bora-3,5,8-trioxatricyclo[7.3.1.04,13]trideca-1(12),9(13),10-trien-6-yl]methyl propanoate hydrogen chloride

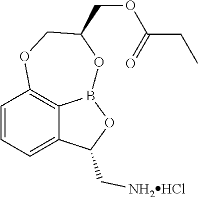

The compound of Example 8 was prepared according to the methods described in US Application US 2013/0165411.

### Utility and Testing

The compounds described herein are boron compounds and prodrugs thereof. The prodrugs will be converted into the parent boron compound in vivo to exert an antibacterial efficacy. Accordingly, the antibacterial activity of the prodrug compounds described herein were tested with the parent boron compound.

### In Vitro Activity of Representative Compounds Against Mycobacteria

The in vitro activity of the parent boron compound described herein was assessed by standard testing procedures such as the determination of minimum inhibitory concentration (MIC) as described in Clinical and Laboratory Standards Institute (CLSI) document M24-A2. Lower MIC values indicate high antibacterial activity, while higher MIC values indicate a reduced antibacterial activity. Generally, MIC values of about ≤2 mg/L indicate a good therapeutic (i.e., suitable for therapy) potency for antibacterial drugs, while MIC values of ≥8 mg/L indicate a lack of therapeutically useful activity for a test compound.

The in vitro activity (potency) of representative compounds described herein against mycobacteria is illustrated by the MIC data of Table 1 below. As evident from the data of Table 1, the compound of Example 4 is highly active against many mycobacteria pathogens, including M. scrofulaceum, M. gordonae, M. avium, M. abscessus, M. intercelleulare, M. fortuitum, Mycobacterium peregrinum, M. smegmatis, M. massiliense. (MIC ranges from 0.063-2 mg/L). In particular, the compound of Example 4 is surprisingly potent against rapidly growing mycobacteria (RGM), including M. abscessus. M. intercelleulare, M. fortuitum, Mycobacterium peregrinum, M. smegmatis, M. massiliense (MIC ranges from 0.063-0.125 mg/L).

The compound GSK656 is a different boron compound disclosed in PCT Application WO/2012/033858, which is generally related to composition provided herein. Surprisingly, despite some structural similarity, the compound of Example 4 and GSK656 display very different antibacterial spectrum against the nontuberculous Mycobacterium. Critically important, Example 4 is highly potent against M. fortuitum, Mycobacterium peregrinum, and M. smegmatis with an value MIC of 0.125 mg/L, while the GSK656 MIC is ≥8 mg/L. Based on these numerical values, the compound GSK656 is more than 64-fold less active than the representative compound of Example 4 provided in the present invention.

Likewise surprising, the compound of Example 4 is also 4-fold more potent than GSK656 against NTM pathogens M. avium and M. intercelleulare. Such vast differences in antibacterial spectrum activity and potency are entirely unexpected. Effectively, the compound of Example 4 possesses greatly and surprisingly improved activity over the reference compound GSK656, and offers the antibacterial coverage against NTM pathogens well beyond that possible for GSK656. Another boron compound, AN2690, disclosed in US patent application US 2006/0234981, also possesses moderate or no activity against all NTM species tested. Due to the complexity of NTM infections, it is the most beneficial and convenient for clinical use when a compound possess activity against a broad antibacterial spectrum to cover many mycobacteria species. Therefore, the composition provided herein offers the best option for treatment of such NTM infections.

TABLE 1
In vitro Antibacterial activity against Mycobacteria pathogens

| | 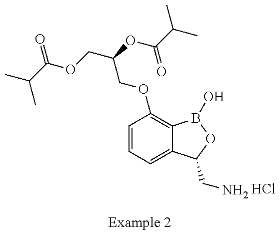 [SMILES: CC(C)C(=O)OC[C@H](COc1cccc2c1B(O)O[C@@H]2CN)OC(=O)C(C)C ; Cl] | 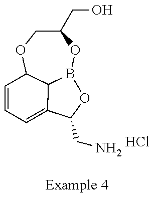 | 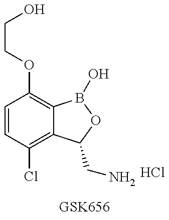 | 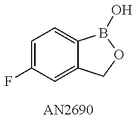 [SMILES: OB1OCc2cc(F)ccc21] |
|---|---|---|---|---|
| Structure | MIC, mg/L | | | |
| M. scro- | / | 0.25 | 4 | 8 |
| fulaceum | | | | |
| M. gordonae | / | 0.5 | 0.5 | 8 |
| M. avium | >8 | 2 | >8 | >8 |
| M. inter- | >8 | 2 | >8 | >8 |
| celleulare | | | | |
| M. abscessus | >8 | 0.063 | 0.063 | >8 |
| M. fortuitum | / | 0.125 | >8 | >8 |
| M. pere- | / | 0.125 | 8 | >8 |
| grinum | | | | |
| M. smegmatis | / | 0.125 | >8 | >8 |
| M. massiliense | / | 0.063 | 0.063 | 4 |

In order to further characterize the antibacterial profile of the compounds described herein, the minimal bactericidal concentration (MBC) was also determined according to Clinical and Laboratory Standards Institute (CLSI) document M24-A2. Both GSK656 and the compound of Example 4 were tested for MIC and MBC against 20 clinical isolates of M. abscessus complex, including M. abscessus and M. massiliense. As shown in Table 2, GSK656 and the compound of Example 4 have similar MIC across the clinical isolates. However, very surprisingly, the compound of Example 4 has a much lower MBC against all isolates tested. An antibiotic was considered bactericidal if the MBC/MIC ratio was <4, or bacteriostatic if the ratio was >4. Therefore, the compound of Example 4 is bactericidal against half of the isolates, while GSK656 is bacteriostatic against all isolates. This beneficial to the compound of Example 4 differentiation is very surprising due to generally similar structure and MIC profile. Indeed, the bactericidal property is very important for a more effective and efficient clearance of the bacterial infection. Specifically, a bactericidal compound kills or completely eradicates the pathogen, while a bacteriostatic compound merely prevents the growth of bacterial. In the latter case, remaining bacteria may develop a bacterial resistance to the agent rendering treatment ineffective, or restart the infection after cessation of initial antimicrobial therapy. Thus, a bactericidal agent is highly preferred to a bacteriostatic agent, especially for eradication of persistent mycobacterial infections.

TABLE 2
MICs and MBCs against clinical
isolates of M. abscessus complex

| | GSK656 | | | Example 4 | | |
|---|---|---|---|---|---|---|
| | MIC | MBC | | MIC | MBC | |
| Clinical | (mg/ | (mg/ | | (mg/ | (mg/ | |
| Isolate | L) | L) | Classification | L) | L) | Classification |
| A215 | 0.063 | 2 | Bacteriostatic | 0.063 | 0.5 | Bacteriostatic |
| A217 | 0.063 | 2 | Bacteriostatic | 0.063 | 0.5 | Bacteriostatic |
| A232 | 0.063 | 1 | Bacteriostatic | 0.063 | 0.125 | Bactericidal |
| A274 | 0.125 | >4 | Bacteriostatic | 0.125 | 2 | Bacteriostatic |
| A35 | 0.063 | >2 | Bacteriostatic | 0.063 | 0.25 | Bactericidal |
| G73 | 0.063 | >2 | Bacteriostatic | 0.063 | 1 | Bacteriostatic |
| G164 | 0.063 | >2 | Bacteriostatic | 0.063 | 0.25 | Bactericidal |
| A51 | 0.063 | >2 | Bacteriostatic | 0.063 | 0.25 | Bactericidal |
| A222 | 0.063 | >> | Bacteriostatic | 0.063 | 0.25 | Bactericidal |
| G74 | 0.063 | >4 | Bacteriostatic | 0.063 | 1 | Bacteriostatic |
| A311 | 0.063 | 1 | Bacteriostatic | 0.063 | 0.25 | Bactericidal |
| G104 | 0.063 | 2 | Bacteriostatic | 0.125 | 0.25 | Bactericidal |
| A213 | 0.063 | 2 | Bacteriostatic | 0.125 | 0.25 | Bactericidal |
| A353 | 0.125 | 2 | Bacteriostatic | 0.125 | 0.25 | Bactericidal |
| A255 | 0.063 | 2 | Bacteriostatic | 0.063 | 0.25 | Bactericidal |
| G87 | 0.063 | >2 | Bacteriostatic | 0.063 | 0.5 | Bacteriostatic |
| G182 | 0.063 | >2 | Bacteriostatic | 0.063 | 1 | Bacteriostatic |
| G110 | 0.063 | >2 | Bacteriostatic | 0.063 | 1 | Bacteriostatic |
| A197 | 0.063 | >2 | Bacteriostatic | 0.063 | 1 | Bacteriostatic |
| A268 | 0.125 | >4 | Bacteriostatic | 0.125 | 2 | Bacteriostatic |

### Activity of Representative Compounds in an M. abscessus Mouse Lung Infection Model

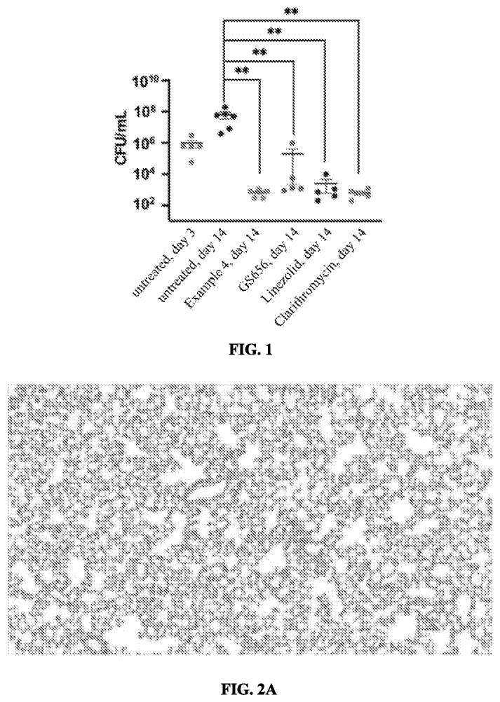

To establish the efficacy of the compounds described herein in vivo, an M. abscessusmouse lung infection model was performed. Groups of BALB/c mice (The mice were randomly divided into groups with 6 mice in each group) were administered cyclophosphamide one week before infection, and then inoculated intranasally with M. abscessus CIP108297 (10⁷ CFU/mouse). Three days after infection, the mice were subsequently treated with 10 mg/kg of the compound of Example 4 or GSK656 administered daily by subcutaneous, or 100 mg/kg linezolid (an approved antibiotic) or 200 mg/kg clarithromycin (an approved antibiotic) administered daily by oral gavage. M. abscessus CFUs in the lungs were quantified at 2 weeks post-infection as shown in FIG. 1. The results showed that the compound of Example 4 caused a significant reduction in bacteria (˜7.8 log 10 CFU) compared to the untreated groups. Moreover, the organisms in the lungs of the mice treated with the compound of Example 4 were reduced markedly compared to animals treated with GSK656. This strikingly improved efficacy is unexpected, considering the similar structure and MIC profile between GSK656 and the compound of Example 4. This may be attributed to the surprising finding of lower MBC in the compound of Example 4. In addition, the compound of Example 4 also showed higher potential for M. abscessus clearance relative to either linezolid or clarithromycin, which were given at very high dosage.

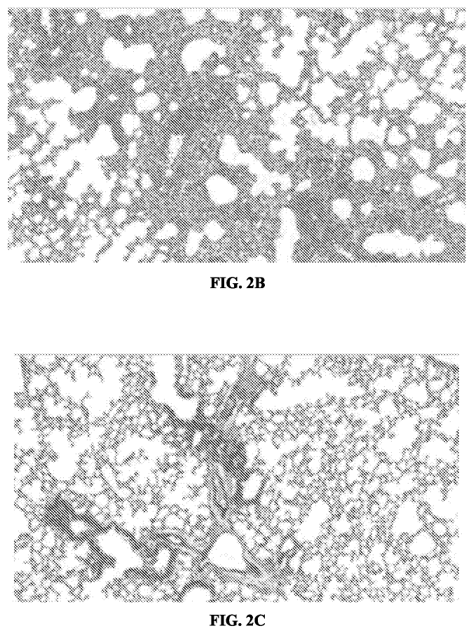

H&E stained tissue sections revealed severe alveolar wall thickening, inflammatory cell infiltration, and diapedesis of the erythrocyte in the lungs of the control group of mice at 2-weeks post-infection. In contrast, pathologic changes were rare and lung lesions were negligible in the group treated with the compound of Example 4 as shown in FIG. 2A-FIG. 2C. These findings demonstrate the efficacy of the compound of Example 4 in inhibiting the replication of M. abscessus in a mouse model of pneumonia.

### Pharmacokinetic Data of Orally-Administered Representative Prodrugs

To further elucidate the therapeutic potential of drug compounds, pharmacokinetic (PK) data is often used to establish key parameters predictive of a therapy outcome, such as the area under the curve (AUC) of a plot monitoring the change in systemic drug concentration over time. A higher AUC value indicates a greater exposure to the drug, commonly associated with a greater therapeutic potential due to a higher amount of drug available to combat the infection. In contrast, a lower AUC value indicates a reduced exposure to the drug, resulting in a reduced amount of antibiotic available to combat bacterial infections. To that end, the compounds described herein were tested in the rat PK model of oral administration performed analogously to the methods described in the monograph Current Protocols in Pharmacology, 2005, 7.1.1-7.1.26, John Wiley & Sons, Inc.

All the compounds were administered to SD rats (The rats were randomly divided into groups with 3 rats in each group) by intravenous or oral gavage. The prodrugs convert to the parent drug molecule in vivo. Therefore, only the parent compound Example 4 was analyzed and quantified in all test samples. As shown in Table 3, the parent compound in Example 4 had moderate oral bioavailability of 15%. The pharmacokinetic data for the prodrugs described herein revealed a greatly improved systemic exposure and Cₘₐₓ at the same dosage of 5 mg/kg. For example, the compound of Example 2 displayed exposure (AUC) and Cmax of 2906 hr*ng/ml and 870 ng/ml, respectively. This unexpected result represents a striking 3.4-fold and 3-fold improvement in the exposure and Cₘₐₓ, respectively, for in vivo drug exposure for the compound of Example 2 compared to the compound of Example 4. The AUC data taken in context of the good efficacy of the compound of Example 4 confirmed in the M. abscessus mouse lung infection model (see above), strongly suggest that the corresponding prodrugs with even higher exposure (AUC) possess further improved therapeutic potential. Importantly, the data indicate that this is achievable by way of an oral administration. Furthermore, based on the known comparison of rodent tests to human data (such as known species-to-species PK scaling), this is possible with a lower dosage for therapy in humans.

TABLE 3
Pharmacokinetic data in rat of representative prodrugs

| | | | | | | Oral |
|---|---|---|---|---|---|---|
| | | Administration | Dosage | AUC | Cₘₐₓ, | bioavail- |
| Compound | Structure | Route | mg/kg | ng*h/ml | ng/ml | ability |
| Example 4 | 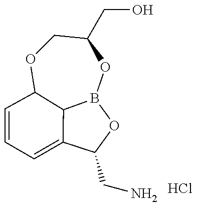 | iv oral | 5 5 | 5661 856 | 6776 139 | / 15% |
| Example 1 | 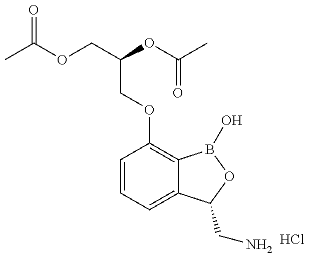 [SMILES: CC(=O)OC[C@H](COc1cccc2c1B(O)O[C@@H]2CN)OC(C)=O ; Cl] | oral | 5 | 1251 | 241 | 22% |
| Example 2 | 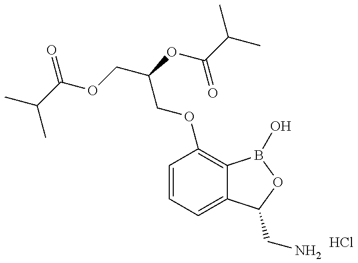 [SMILES: CC(C)C(=O)OC[C@H](COc1cccc2c1B(O)O[C@@H]2CN)OC(=O)C(C)C ; Cl] | oral | 5 | 2906 | 870 | 51% |
| Example 5 | 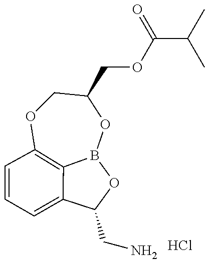 [SMILES: CC(C)C(=O)OC[C@@H]1COc2cccc3c2B(O1)O[C@@H]3CN ; Cl] | oral | 5 | 2232 | 581 | 39% |
| Example 6 | 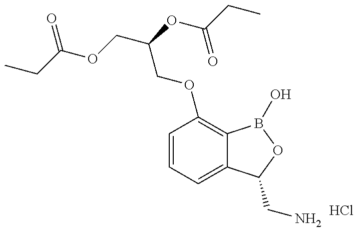 [SMILES: CCC(=O)OC[C@H](COc1cccc2c1B(O)O[C@@H]2CN)OC(=O)CC ; Cl] | oral | 10 | 3113 | / | 27% |
| Example 7 | 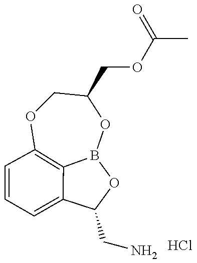 [SMILES: CC(=O)OC[C@@H]1COc2cccc3c2B(O1)O[C@@H]3CN ; Cl] | oral | 10 | 3944 | / | 30% |
| Example 3 | 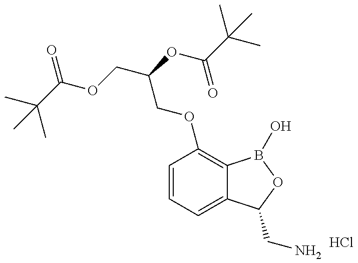 [SMILES: CC(C)(C)C(=O)OC[C@H](COc1cccc2c1B(O)O[C@@H]2CN)OC(=O)C(C)(C)C ; Cl] | oral | 10 | 3279 | / | 29% |
| Example 8 | 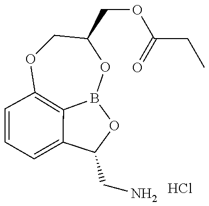 [SMILES: CCC(=O)OC[C@@H]1COc2cccc3c2B(O1)O[C@@H]3CN ; Cl] | oral | 10 | 5421 | / | 38% |

Moreover, in a lung distribution study conducted in Balb/C mouse (three mice per time point), Example 2 showed much higher exposure in the lung than in the plasma (assessed by the area under the lung/plasma concentration time curve (AUC). As shown in Table 4, compounds of Example 4 and 2 were administered at 10 mg/kg via iv and oral, respectively. In analysis of Example 2, the concentration of parent compound (Example 4) and prodrug (Example 2) were determined in both plasma and lung. The prodrug (Example 2) were rapidly converted to Example 4, with little prodrug can be detected in plasma. The oral bioavailability of Example 4 generated from Example 2 is 83.95% in mice, compared to the AUC of Example 4 administered by iv. Despite the rapid conversion of the prodrug, it is very surprised to see more Example 4 were detected in lung, with a lung/plasma AUC ratio of 5.24, which is almost 2.2-fold higher than that of Example 4 administered by iv.

TABLE 4
Lung versus plasma distribution of Example 4 and 2 in mice

| | Example 4 | | Example 2 | | | |
|---|---|---|---|---|---|---|
| | 10 mg/kg, iv | | 10 mg/kg, po | | | |
| | Example 4 | | Example 4 | | Example 2 | |
| | plasma | lung | plasma | lung | plasma | lung |
| t_{1/2}, h | 1.87 | 1.46 | 1.93 | 2.09 | NA | 6.42 |
| AUCₗₐₛₜ | 5359 | 12822 | 4499 | 23589 | 0.0421 | 968 |
| | ng*h/ml | ng*h/g | ng*h/ml | ng*h/g | ng*h/ml | ng*h/g |

| AUC ratio | 2.39 | | 5.24 | / | |
|---|---|---|---|---|---|
| (lung/plasma) | | | | | |
| Oral | / | / | 83.95% | / | / |
| bioavailability | | | | | |

Above representative data taken in its entirety reveal a surprisingly superior therapeutic potential for the compounds invented herein, with the beneficial unexpected advantages in areas of potency, efficacy, and exposure not anticipated from any prior patents or publications on boron anti-infectives. The dramatic and surprising improvement in distinctly different critical parameters for antibacterial compounds provided herein offers marked benefits for human or mammal therapy, including but not limited to improved bactericidal activity, superior in vivo efficacy, convenient oral administration for long therapy duration, and reduced possible adverse effects.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While embodiments have been described herein in conjunction with a preferred aspect, a person with ordinary skills in the art, after reading the foregoing specification, can affect changes, substitutions of equivalents and other types of alterations to the embodiments as set forth herein. Each aspect described above can also have included or incorporated therewith such variations or aspects as disclosed in regard to any or all of the other aspects. The description herein is also not to be limited in terms of the particular aspects described herein, which are intended as single illustrations of individual aspects provided herein. Many modifications and variations described herein can be made without departing from its spirit and scope, as will be apparent to those skilled in the art. Functionally equivalent methods within the scope of this description, in addition to those enumerated herein, will be apparent to those skilled in the art from the foregoing descriptions. It is to be understood that this description is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A method for treating a nontuberculous mycobacterial infection comprising administering to a mammal in the need thereof a compound of Formula I: 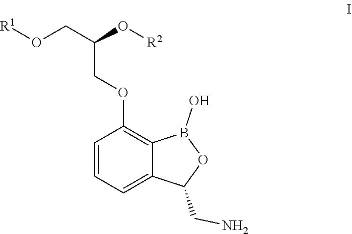
or a pharmaceutically acceptable salt or a pharmaceutical composition thereof;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; and
R² is selected from C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl; or
R¹ and R² taken together form a heterocyclic group selected from 1,3-dioxane, 2-C₁₋₆alkyl-1,3-dioxane, 2,2-di(C₁₋₆alkyl)-1,3-dioxane, 2-methyl-1,3-dioxane, 2-aryl-1,3-dioxane, 2-(2-carboxyphenyl)-1,3-dioxane, 2-(4-carboxyphenyl)-1,3-dioxane, and 2-C₁₋₆alkylOC(═O)-1,3-dioxane; each of which is optionally substituted with one to four R³;
R³ at each occurrence is independently selected from the group consisting of halo, hydroxy, C₁-C₆ alkyl, C₃-C₆ cycloalkyl, C₁-C₆ alkoxy, aryl, and heteroaryl; or
two R³ groups when attached to adjacent carbons are taken together with the carbons to which they are attached to form a fused C₃-C₆ cycloalkyl; or
two R³ groups when attached to the same carbon are taken together with the carbon to which they are attached to form a spiro C₃-C₆ cycloalkyl;
wherein each R³ is optionally independently substituted with one to three halo, hydroxy, or C₁-C₃ alkyl;
and wherein the nontuberculous mycobacteria is selected from Mycobacterium scrofulaceum, Mycobacterium gordonae, Mycobacterium avium, Mycobacterium abscessus, Mycobacterium intercelleulare, Mycobacterium fortuitum, Mycobacterium peregrimim, Mycobacterium smegmatis, and Mycobacterium massiliense.

2. A method for treating a nontuberculous mycobacterial infection comprising administering to a mammal in the need thereof a compound of Formula II: 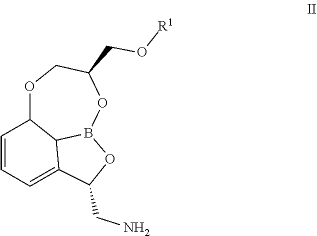
or a pharmaceutically acceptable salt or a pharmaceutical composition thereof;
wherein:
R¹ is selected from H, C₁₋₂₄alkyl-C(═O)—, C₁₋₂₄alkoxy-C(═O)—, C₃₋₇cycloalkyl-C(═O)—, heteroalkyl-C(═O)—, aryl-C(═O)—, heteroaryl-C(═O)—, and (5-methyl-1,3-dioxol-2-one-4-yl)methyl;
and wherein the nontuberculous mycobacteria is selected from Mycobacterium scrofulaceum, Mycobacterium gordonae, Mycobacterium avium, Mycobacterium abscessus, Mycobacterium intercelleulare, Mycobacterium fortuitum, Mycobacterium peregrimim, Mycobacterium smegmatis, and Mycobacterium massiliense.

3. The method according to claim 1, wherein R¹ is C₁₋₄alkyl-C(═O)— and R²is C₁-4alkyl-C(═O)—.

4. The method according to claim 1, wherein the compound is selected: 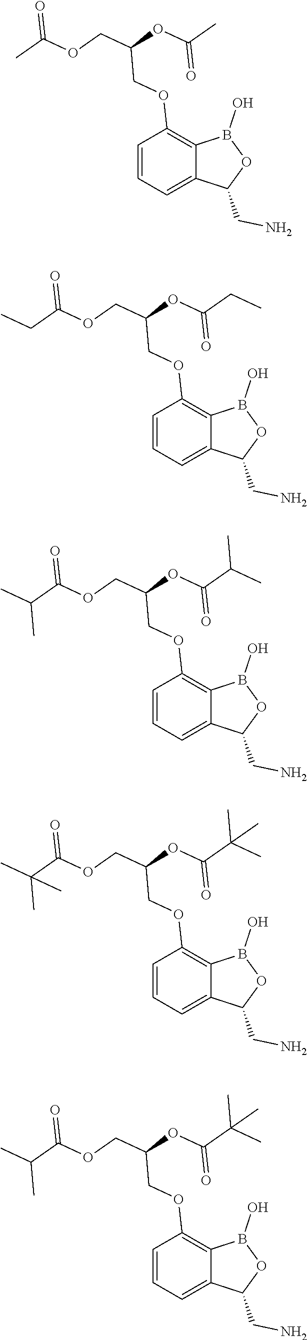 [SMILES: CC(=O)OC[C@H](COc1cccc2c1B(O)O[C@@H]2CN)OC(C)=O ; CCC(=O)OC[C@H](COc1cccc2c1B(O)O[C@@H]2CN)OC(=O)CC ; CC(C)C(=O)OC[C@H](COc1cccc2c1B(O)O[C@@H]2CN)OC(=O)C(C)C ; CC(C)(C)C(=O)OC[C@H](COc1cccc2c1B(O)O[C@@H]2CN)OC(=O)C(C)(C)C ; CC(C)C(=O)OC[C@H](COc1cccc2c1B(O)O[C@@H]2CN)OC(=O)C(C)(C)C] 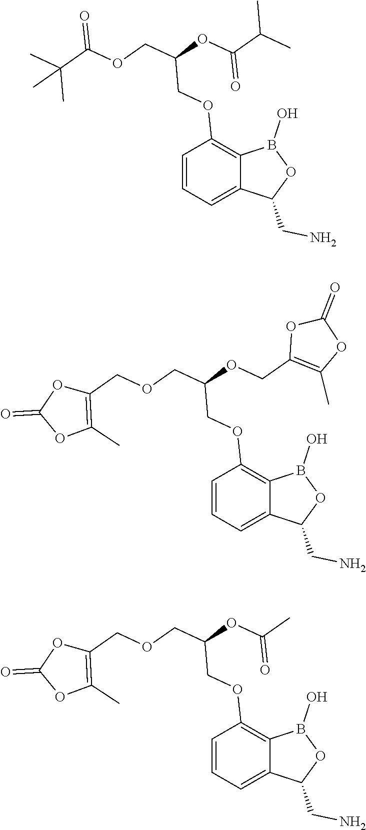 [SMILES: CC(C)C(=O)O[C@H](COC(=O)C(C)(C)C)COc1cccc2c1B(O)O[C@@H]2CN ; Cc1oc(=O)oc1COC[C@H](COc1cccc2c1B(O)O[C@@H]2CN)OCc1oc(=O)oc1C ; CC(=O)O[C@H](COCc1oc(=O)oc1C)COc1cccc2c1B(O)O[C@@H]2CN]
or a pharmaceutically acceptable salt or a pharmaceutical composition thereof.

5. The method according to claim 2, wherein the compound is selected from: 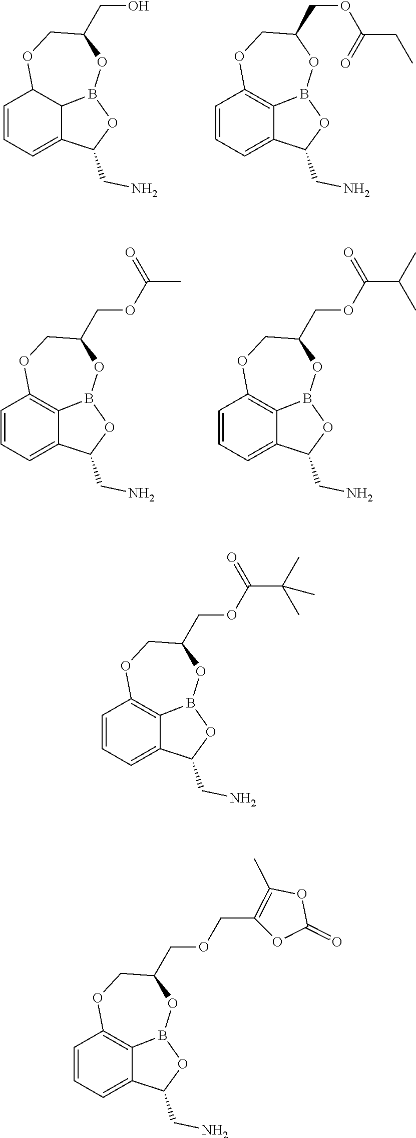
or a pharmaceutically acceptable salt or a pharmaceutical composition thereof.

6. The method according to claim 1, wherein the pharmaceutically acceptable salt is a hydrochloride salt.

7. The method according to claim 1, wherein the compound or pharmaceutically acceptable salt thereof is administered to the orally, parenterally, intraperitoneally, intravenously, intraarterially, transdermally, sublingually, intramuscularly, rectally, transbuccally, intranasally, via inhalation, vaginally, intraoccularly, via local administration, subcutaneously, intraadiposally, intraarticularly, intraperitoneally or intrathecally.

8. The method according to claim 1, wherein the compound or pharmaceutically acceptable salt thereof is administered once or twice per day in the form of a single dosage in a range of 10-1000 mg.

9-11. (canceled)

12. The method according to claim 1, wherein the infection is a skin, soft tissue, respiratory, blood, intra-abdominal, urinary, bone, or eye infection.

13. The method according to claim 1, wherein the compound or pharmaceutical composition is administrated for 4-12 months.

14. The method according to claim 1, wherein the compound or pharmaceutically acceptable salt thereof or pharmaceutical composition thereof, is administered in a dosage form suitable for oral administration.

15. The method according to claim 1, wherein the compound, or pharmaceutically acceptable salt thereof or pharmaceutical composition thereof, is administered in a dosage form suitable for oral administration, and the compound exhibits an oral bioavailability from about 50% to about 100%.

16. The method according to claim 2, wherein the pharmaceutically acceptable salt is a hydrochloride salt.

17. The method according to claim 2, wherein the compound or pharmaceutically acceptable salt thereof is administered to the orally, parenterally, intraperitoneally, intravenously, intraarterially, transdermally, sublingually, intramuscularly, rectally, transbuccally, intranasally, via inhalation, vaginally, intraoccularly, via local administration, subcutaneously, intraadiposally, intraarticularly, intraperitoneally or intrathecally.

18. The method according to claim 2, wherein the compound or pharmaceutically acceptable salt thereof is administered once or twice per day in the form of a single dosage in a range of 10-1000 mg.

19. The method according to claim 2, wherein the infection is a skin, soft tissue, respiratory, blood, intra-abdominal, urinary, bone, or eye infection.

20. The method according to claim 2, wherein the compound or pharmaceutical composition is administrated for 4-12 months.

21. The method according to claim 2, wherein the compound or pharmaceutically acceptable salt thereof or pharmaceutical composition thereof, is administered in a dosage form suitable for oral administration.

22. The method according to claim 2, wherein the compound, or pharmaceutically acceptable salt thereof or pharmaceutical composition thereof, is administered in a dosage form suitable for oral administration, and the compound exhibits an oral bioavailability from about 50% to about 100%.
